(19) United States
(12) Patent Application Publication
(10) Pub. No.: US 2009/0240299 A1
(43) Pub. Date: Sep. 24, 2009

(54) DEVICE AND METHOD FOR REFLEX CARDIAC PACING

(76) Inventors: Bunmi T. Adekore, Arlington, MA (US); Peter L. Gallagher, Lexington, KY (US); Felix O. Sogade, Macon, GA (US)

Correspondence Address: LANDO & ANASTASI, LLP, ONE MAIN STREET, SUITE 1100, CAMBRIDGE, MA, 02142, US

(21) Appl. No.: 12/051,575

(22) Filed: Mar. 19, 2008

(51) Int. Cl. A61N 1/365; A61N 1/36

(52) U.S. Cl. 607/14; 607/27; 607/36

(57) ABSTRACT

Solid state piezoelectric or Lorentzian components are utilized to generate electrical energy in an implanted device. The energy generated from tissue displacement is stored and made available for use as a cardiac pacing charge to be delivered by the device when a triggering condition, such as an arrhythmia is detected. A plurality of implanted devices can be used to collectively provide one or more pacing charges.

### BACKGROUND OF INVENTION

1. Field of Invention

This invention relates to the development of piezoelectric and Lorentzian nano- and micro-dimensional materials and associated logical systems for arrhythmia pacing.

2. Discussion of Related Art

The cardiac electrical system is a complex electrophysiologic circuit. The components of the human cardiac conduction system typically begin with the electrical impulse generating site in the right atrium called the sino-atrial node (SN). The propagating electrical wave front passes through specialized interatrial myocardial fibers, such as Bachmann's bundle (BB), to activate the left atrium. In order for the ventricles to be activated, the electrical wave front passes through the atrio-ventricular (AV) node. After exiting the AV node, electricity passes through the Bundle of His, rapidly activating the right ventricle via the right bundle branch (RBB) and the left ventricle through the left bundle branch (LBB). The LBB is composed of the left anterior and posterior fascicular systems terminating in the Purkinje fibers. The ventricles are activated synchronously with simultaneous activation of the ventricular septum and lateral walls. The electrical activation of the ventricles is complex and involves the coordinated contraction of different myocardial layers resulting in clockwise (systolic) and counterclockwise (diastolic) torsional twist.

Disorders of the human cardiac conduction system can occur at one or all of the cardiac circuit. Impairment of the components of the conduction system may result in bradyarrhythmias such as abnormally slow or non-existent beats (SN), abnormal inter-atrial conduction (BB), or intermittent or complete loss of conduction to the ventlicles (A V node, His Bundle, Purkinje fibers). Abnormalities in the synchronous activation of the ventricles may lead to congestive heart failure (CHF). Damage or abnormal activation of the RBB, LBB and/or Purkinje fibers may result in impaired clockwise torsional twist and result in systolic CHF. Delayed or abnormal activation of counterclockwise twist may result in impaired counterclockwise torsional twist and result in diastolic CHF.

Current permanent pacemakers consist of an external generator. The pulse generator is the attached to leads, which are advanced through the venous vascular system to the appropriate cardiac chamber. Leads are attached both passively (with tines) and actively (extendable-retractable screw) affixed to the myocardium. The generator produces an electric impulse which results in the initiation and propagation of contraction. The pacemaker generator delivers an electrical impulse of typically 1 volt-sec through electrodes to the patient's chest as in the case of transcutaneous pacing or to the chambers of the myocardium as in the case of transvenous and permanent pacing. Permanent pacing employs an implantable pacemaker impregnated within a matrix which is inert to the immune system of the body such as titanium, and consists of a macroscale (dimensions exceeding 1 cm²) generator or power source, programmable logic circuits, and electrical leads to the myocardium from a generator/power source. Advances in implantable pacemaker technology have led to the incorporation of microprocessor for the detection and stimulation of heart rate as well as pacing of not only ventricular chambers but the atria as well.

Tachy-arrhythmias are disorders of the electrical conduction system which may result in inappropriately fast and/or dangerous beats. Current implantable cardiac defibrillators (ICD) primarily treat dangerous ventricular arrhythmias by detecting the arrhythmia and automatically delivering a monophasic or biphasic shock between the coils of the ICD lead and the ICD generator. In addition, rapid pulse delivery may terminate a ventricular arrhythmia.

Implantable pacemakers typically involve considerations regarding their macroscale dimensionality. Large devices typically require significant invasive surgery for the placement of these devices. The dimensions are also well correlated with the dimension of the generators or power sources required to deliver requisite electrical impulses for pacing. Thus, it is may be advantageous to furnish the requisite electrical impulses over much reduced area and volume dimensions.

Implantable pacemakers can be associated with bulky electrical leads associated with the delivery of electrical impulses. These leads are typically communicated from the regions below the subcutaneous layer of the chest but above the chest skeletal cavity through the myocardium. In many cases, potential or definite and re-occurring bacterial infections associated with electrical lead retention can occur in patients, sometimes resulting in death. Moreover, cases of lead replacement can require additional invasive surgery which is sometimes complicated by inability to locate the leads. Thus, it may be advantageous to minimize transmission distance and dimensions of electrical leads.

Further, potential damage to the leads of implantable pacemakers can result from crush injury as the leads travel in between the clavicle and chest wall. This can be a notable concern for both the short and long term durability of these leads. Further, long leads can have considerations associated with constant motion and subsequent wear and tear. This constant motion may result in cardiac perforations, micro-perforations, dislodgements and micro-dislodgents, lead fractures and abnormal-inconsistent pacing and sensing. Current generators and leads are limited in the number of coordinated sites of sensing and activation.

Also, existing implantable generator and lead systems are limited to only measuring voltage, lead impedance, minute ventilation and generator movement-activity. No current systems can independently measure tip movement-position, blood flow, turbulence or pressure.

### SUMMARY OF THE INVENTION

Some aspects of the invention are directed to implantable cardiac devices. In some embodiments thereof, the cardiac device can comprise a charging circuit configured to store electrical energy generated from at least one of a piezoelectric material and a magnetostrictive material, and a pulse-generating circuit operatively coupled to the charging circuit and configured to deliver at least a portion of the stored electrical energy in at least one electrical pacing charge to at least one cardiac chamber. The charging circuit, in some cases, can comprise at least one storage device; in particular, the at least one storage device can comprise at least one capacitor. The charging circuit can comprise at least one electrical generator having a cantilever element comprising the at least one of the piezoelectric and magnetostrictive materials operatively engaged with a rigid element. In some further embodiments of the invention, the implantable cardiac device can further comprise a sensing circuit configured to monitor cardiac activity of at least one cardiac chamber, and energize the pulse-generating circuit to deliver the at least one electrical pacing charge to the at least one cardiac chamber when the monitored cardiac activity comprises at least one arrhythmic cardiac condition. In still further embodiments, the at least one arrhythmic cardiac condition is a condition selected from the group consisting of tachy-arrhythmia, brady-arrhythmia, abnormal interatrial conduction, and asynchronous ventricular activation. In still other embodiments of cardiac devices of the invention, the at least one of the piezoelectric and magnetostrictive materials comprises a compound selected from the group consisting of zinc oxide, gallium nitride, cuprates, titanates, related alloys, and mixtures thereof.

Other aspects of the invention are directed to a method of electrophysiologic stimulation. In some embodiments pertinent to such aspects of the invention, the method of electrophysiologic stimulation can comprise identifying a patient susceptible to arrhythmic cardiac pacing, and implanting a cardiac pacing device in the patient, the cardiac pacing device comprising at least one charging circuit configured to store electrical energy generated from at least one of a piezoelectric and a magnetostrictive material, and a pulse-generating circuit configured to deliver at least a portion of the stored electrical energy in at least one electrophysiologic stimulating charge to at least one cardiac chamber. The cardiac pacing device can comprise a sensing circuit configured to monitor, at least partially, cardiac activity of at least one cardiac chamber and energize the pulse-generating circuit to deliver the at least one electrophysiologic stimulating charge. In some advantageous embodiments of the methods of the invention, the sensing circuit can be configured to energize the pulse-generating circuit when at least one arrhythmic cardiac condition selected from the group consisting of tachy-arrhythmia, brady-arrhythmia, abnormal inter-atrial conduction, and asynchronous ventricular activation is identified. The charging circuit can, in some cases, comprise at least one generator, each of which can have at least one oscillating member. The oscillating member can be comprised of the piezoelectric and/or the magnetostrictive material. The generator can also have at least one stationary member, typically correspondingly engaged with one or more oscillating members, and at least one dielectric material, typically disposed between the engaged oscillating and stationary members. Implanting the cardiac pacing device can comprise securing the oscillating member to a first tissue region of a pulsating organ and securing the stationary member to a second tissue region of the pulsating organ. In some advantageous embodiments of the invention, the method can comprise implanting a plurality of the cardiac pacing devices in the patient.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing.

In the drawings:

FIG. 1 illustrates an apparatus in accordance with one or more embodiments of the invention;

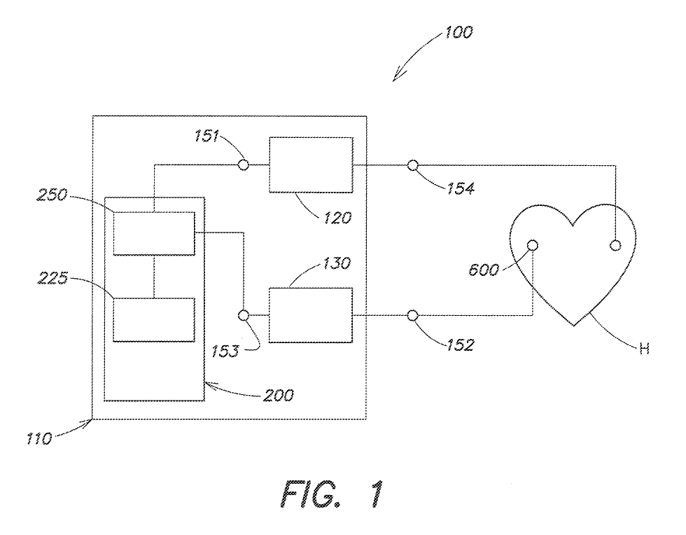

FIG. 2 schematically illustrates components or subsystems of the apparatus FIG. 1 in accordance with one or more embodiments of the invention;

FIGS. 3A and 3B schematically illustrate generating systems in accordance with some embodiments of the invention;

FIGS. 4A and 4B schematically illustrate another embodiment of a generator in accordance with some aspects of the invention, showing the generator static (FIG. 4A) and actuated (FIG. 4B);

FIGS. 5A and 5B schematically illustrate assemblies of generators schematically presented in FIGS. 3A and 3B (FIG. 5A) and in FIGS. 4A and 4B (FIG. 5B) in accordance with some embodiments of the invention;

FIG. 6 schematically illustrates electrodes in contact or interfaced with tissue in accordance with some embodiments of the invention;

FIG. 7 illustrates a circuit representative of the devices pertinent to some embodiments of the invention;

FIG. 8 illustrates a circuit representative of some devices pertinent to some embodiments of the invention; and

FIG. 9 illustrates a plurality of circuits representative of interconnected structures of the invention.

### DETAILED DESCRIPTION

The invention can provide implantable devices that can generate and store electrical energy. The devices and components thereof can, preferably, deliver the stored electrical energy to provide corrective treatment. The invention can involve power sources or generators that have high electrical efficacy and minimum area and volume dimensions that are at least partially associated with piezoelectric and/or ferroelectric nanoscale or microscale structures. Another aspect of the invention provides minimal transmission distance for electrical leads ranging from nanoscale and microscale dimensions to mitigate exposure to bacterial infections due to lead retention and also to lead failure. An aspect of the invention pertains to the affixation method of the novel devices or components thereof to, for example, the myocardium or other tissue. The devices can also be affixed to and provide treatment to damaged areas of the brain for stimulation of affected brain tissue, spinal cord tissue as well as other parts of the central nervous system, in muscle beds to, for example, stimulate contractility in patients with muscular diseases such as, but not limited to, muscular dystrophy and amyotrophic lateral sclerosis. Other applications can involve treatment of patients with gastrointestinal motility disorders such as, but not limited to, achalasia, atonic colon, gastric paresis in diabetics. Still further applications can involve implantation and treatment in fetuses or new born babies with congenital complete heart block conditions. The devices and techniques of the invention can also be utilized in pediatric populations where implanted leads have to be replaced as the patient grows. Thus, the devices and techniques of the invention may provide embedded systems that can obviate future procedures. Further, the devices and techniques of the invention may provide stimulation of the carotid sinus to provide effective treatment for essential hypertension.

The invention can also pertain to the development of non-invasive or minimally invasive delivery techniques of the disclosed devices. Delivery systems can be adapted for certain regions of the body. For example, laparoscopic techniques may be utilized for delivery to the gastrointestinal system and pelvis, whereas endovascular approaches will most likely be the preferred mode for the cardiac applications. Where open chest procedures are available, epicardial placement of the devices of the invention may be preferred.

The invention contemplates multiple implantable devices in any of the herein described applications. Further, the invention can involve devices comprising optoelectronic components that facilitate intra-device communication thereby facilitating, for example, coordinated cardiac stimulation or pacing involving multiple devices. Operative linkages providing intra-device communication may also be effected by radio or other wireless communication techniques.

The invention can also pertain to devices as exemplarily described herein having any one or more of position sensors, flow sensors, or pressure-turbulence sensors.

With reference to the drawings, some aspects of the invention are directed to pacing devices 100, e.g., implantable reflexive cardiac pacing devices, comprising, in some embodiments, at least one charging system or circuit 200 configured to generate and, preferably, to also store electrical energy generated from at least one of a piezoelectric and a magnetostrictive or Lorentzian material that can utilize Lorentz generated electromotive forces, and at least one pulse-generating system or circuit 120, typically operatively coupled to charging circuit 200, and configured to deliver at least a portion of the electrical energy 151 stored therein in at least one electrical pacing charge 154 to at least one cardiac chamber of a heart H.

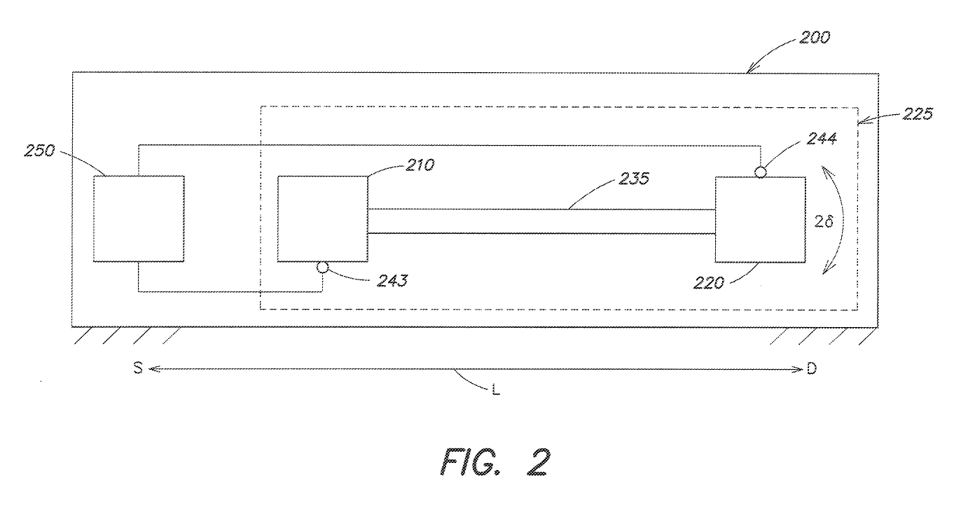

The at least one charging circuit 200, in some cases, comprises at least one storage device 250. The at least one energy storage device 250 can comprise at least one capacitor, battery, or combinations thereof. In accordance with some advantageous embodiments of the invention, the at least one charging circuit 200 can comprise at least one electrical generator 225 or assembly, each having at least one charge generating element, exemplarily illustrated in FIG. 2 as a piezoelectric and/or Lorentzian cantilevered structure 235, operatively engaged with a rigid or fixed element 210 at a first end and a dynamic element 220 at a second end.

In some embodiments of the invention, the implantable cardiac device can further comprise at least one sensing system or circuit 130 configured to monitor cardiac activity 152 of the at least one cardiac chamber and at least one triggering system or circuit 140 that can provide at least one triggering signal 153 to at least one of charging circuit 200, storage device 250, and pulse generating circuit 120. In preferred configurations, the at least one sensing circuit 130 can be operatively coupled to energize the at least one pulse-generating circuit 120 to deliver at least one electrical pacing charge 154 to the at least one cardiac chamber when a triggering condition or signal 152 is detected. The at least one monitored activity that creates a triggering condition can comprise at least one arrhythmic cardiac condition. In still further embodiments, the at least one arrhythmic cardiac condition is a condition selected from the group consisting of tachy-arrhythmia, brady-arrhythmia, abnormal interatrial conduction, and asynchronous ventricular activation.

The materials of the invention can be utilized to generate cumulative electrical potential energy sufficient to facilitate corrective arrhythmic disorders or pace the heart. For example, the devices of the invention can provide one or more electrical stimuli that correct failure which results when the impulse generated by the heart's biological pacemaker, e.g., the sinoatrial node is too slow or fails to travel to the ventricles. The techniques and devices of the invention can also be utilized for sub-threshold stimulation of the heart to prevent certain arrhythmias whereby the heart beats dangerously too fast. Additionally, pacing at different regions of the heart or at multiple sites can enable weakened heart muscles regain some lost functionality through the delivery of electric charge generated by the nano- and/or micro-scale materials of invention.

The oscillating or displacing end of, for example, structure 235, illustrated as being attached or secured to dynamic member 220, can be secured to a dynamic or displaceable position D, thereby providing a stress on structure 235 that preferably results in an induced strain that generates an electric field in the piezoelectric material of structure 235. In such configurations, member 220 is typically affixed to the position D through the case or body of generator 200. The resultant displacement can be expressed as a relative change or difference of dimension, e.g., planar along a longitudinal axis of structure 235, measured from position D to position S.

Other energizing effects can result from one or more angular or bending distortions, represented as displacement 26 at an end of structure 235 adjacent to member 220, at a normal direction relative to the longitudinal axis of structure 235. Such displacements may occur at a plurality of axes normal to the longitudinal axis of structure 235.

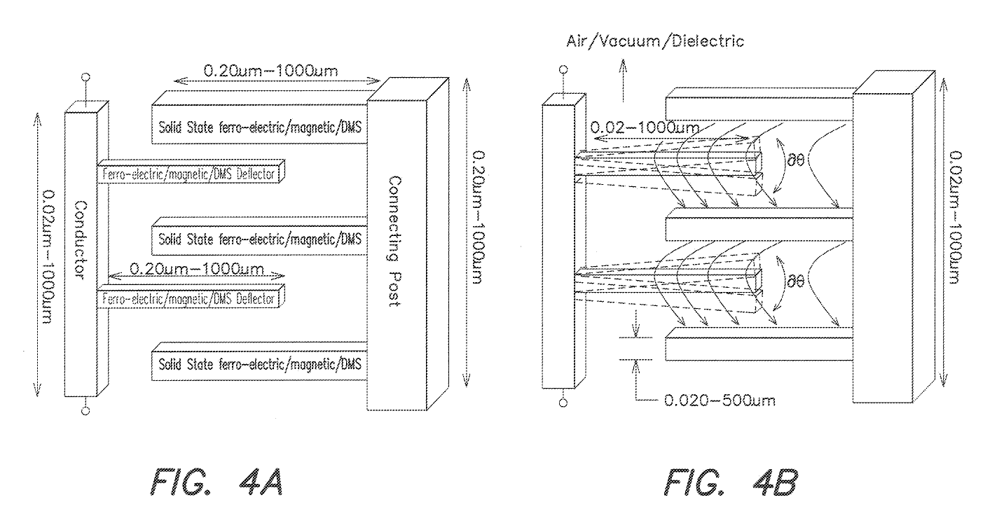

The nanoscale or microscale generators of the invention typically have dimensions that facilitate amplitudinal responses. For example, the length of structure 235 can be at least twice as long as its width in a configuration that is aligned or configured to be within the range of the natural frequency of oscillating or pulsating tissue, e.g., the heart, which is typically defined between about 0.9 to about 1.2 Hz, depending on the patient. These configurational congruences advantageously facilitate utilization of the mechanical movement or displacement ∂θ defined as deflections within the angular range 0<∂θ<90° of a dynamic or oscillating member that has an end fixed or secured to a stationary member at stationary position as depicted in FIGS. 4A and 4B, discussed further below.

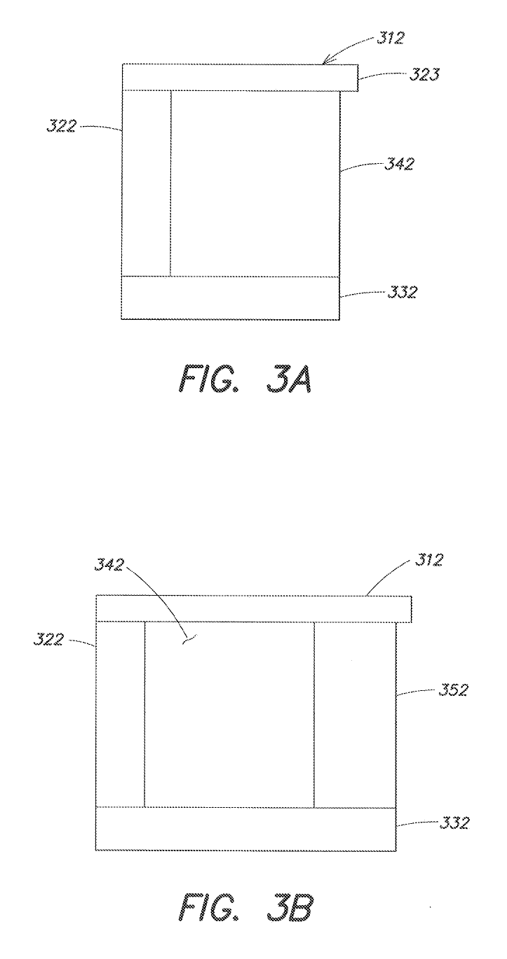

The dimensions of the piezoelectric or ferroelectric components are typically in the microscale or, preferably, in the nanoscale regime. Such displacement reliant energy-generating structures can have an aspect ratio that provides mechanical oscillations based on a cantilever design in which the piezo- and/or ferro-electric components 312 extend from a post 322 on which it rests and in which mechanical deflections from the zero or rest position of the cantilevered member is facilitated through the pulsation of the heart or another muscle as shown in FIGS. 3A and 3B. The deflection of an unsecured end 323 of piezoelectric component 312 typically generates the harvested electrical energy stored in storage 250. Deflection of end 323 can be along any axis normal to the longitudinal axis of component 312.

In another embodiment, cantilever 312 can comprise a conducting or non-conducting material or can comprise a piezo- or ferro-electric material that can communicate a stress or force sufficient to, preferably, elastically compress a piezoelectric and/or a ferroelectric nanoscale and/or microscale structure 352 disposed between cantilever 312 and relatively fixed substrate 332 as shown in FIG. 3B. Such configurations can advantageously be utilized where the applied stress is sufficient to deform cantilever 312 and structure 352. The transferred stress can thus generate the strain that facilitates generation of the storable electric energy from the piezoelectric or Lorentzian material that can comprise structure 352. The structures can be separated from the post and/or substrate by air or vacuum or a dielectric material 342. Where a plurality of generators are utilized, these assemblies can collectively be subjected to mechanical deflection or deformation and can be connected to a conducting or non-conduction post as shown in FIGS. 4A and 4B.

Generator 200 can comprise one or more cantilever structures 235, comprising piezoelectric or Lorentzian materials, secured at a first end to a member 210 which, preferably is secured to tissue at any of positions S and D, or both, that is pulsating or has a natural harmonic behavior with a driving frequency, ω. At a second end distal from the first end secured to member 210, cantilever structure 235 can be secured a member 220 having a mass, m. The natural oscillations of the tissue at any of positions S, D, or both, can be transformed into deflection 2δ, of piezoelectric member 220, which in turn typically generates electrical energy conducted through leads 243 and 244 to storage 250. Thus, for example, cantilever structure 235 and member 220 can be sized and constructed to have a resonance frequency, ω₀, that preferably corresponds to the natural driving frequency ω of the tissue, based on the relationships:

$$δ = 3 \frac{σ ( 1 - υ )}{E} {( \frac{{L}_{0}}{t} )}^{2}$$

$$k = \frac{{Ewt}^{3}}{4 {L}_{0}^{3}} = \frac{F}{δ} , \\ {ω}_{0} = \sqrt{\frac{k}{m}} ,$$

where δ is the free end deflection of cantilever structure 235, e.g., at member 220; σ is the applied stress; and E is the Young's modulus, L₀ is the length, w is the width, t is the width, and m is the effective mass of the structure, v is Poisson's ratio of the cantilever material, and k is the spring constant of the structure.

In some cases, the one or more generators typically have dimensions that facilitate amplitudinal responses to the natural frequency of the pulsating tissue, e.g. the heart, that can provide mechanical movements or deformations that induce an electromagnetic current within structures residing within a magnetic or dilute magnetic field. The dimensions of the ferroelectric and/or magnetic nanoscale and/or micro-structures can have an aspect-ratio enabling mechanical oscillations or deflections from its rest position within a magnetic matrix in response to the natural frequency of the heart as shown in FIGS. 4A and 4B. The mechanical deformations facilitate Lorentz or electromagnetic induction phenomena. For example, the dimensions of the ferroelectric and/or magnetic nanoscale and/or microscale structures provide an aspect-ratio enabling mechanical oscillations or deflections from its rest position within a magnetic matrix in response to the natural frequency of, for example, the heart as shown in FIG. 4B.

Mechanical displacement of the dynamic member 220 relative to the fixed member 210 is typically translated into electrical energy by the piezoelectric or ferroelectric cantilevered member or storage structure 250 or generator 225. The generated potential can be represented by the relationships:

$${V}_{peak} ≺ \frac{Δ L}{L} \cdot {E}_{ij}$$

$$ξ = - N \frac{d {Φ}_{B}}{d t}$$

where Eᵢⱼ is the piezoelectric field tensor of the piezoelectric members, measured in Volts/per unit length of the member and Vₚₑₐₖ is the peak voltage generated at peak or maximum deflection measured in Volts, ΔL is change in length of the piezoelectric member from a state of zero deformation to a state wherein measurable elastic deformation or deflection occurs, L is the initial or undeformed length of the piezoelectric member, N is the number of fixed electromagnets or permanent magnets, Φ_{B} is magnetic flux measured in weber, volt-second or tesla, and t is time measured in seconds.

If a Lorentzian material is utilized, a magnetic field would be created by the oscillatory displacement of the dynamic element relative to the stationary element. A coil (not shown) can be disposed to translate the magnetic energy into electrical energy which can then store in the one or more storage systems. In another configuration, the cantilever structure can comprise a Lorentzian or ferroelectric. Typically, the cantilevered structures are constructed based on the force deflection relationships presented above. In particular embodiments, the cantilevered structures can have an aspect ratio that allows angular deflections. In further particular embodiments, the structures can have a length that is at least twice as long as its width, is suspended between one or two or multiple fixed or stationary members which possess measurable electromagnetic or permanent magnetization such that a magnetic field is created by these fixed members and wherein the angular deflections of the cantilever member within this magnetic field due to oscillations at the natural frequency of the heart generates an electromotive force and whereby the electromotive force generated thereafter charges a solid state capacitor. Non-limiting examples of such materials include alloys of terbium, iron and dysprosium such as Terfenol-D.

The energy generating materials and components of the invention can be based on a group of compounds including, but not limited to, zinc oxide, gallium nitride, and related alloys thereof such as ZnₓCo₁₋ₓO, ZnₓMn₁₋ₓO, GaₓMn₁₋ₓN; metallic cuprates and titanates such as, but not limited to, yttrium cuprate (YtₓCu₁₋ₓO_{y}), niobium titanate (NbₓTi₁₋ₓO_{y}), and nickel titanate (NiₓTi₁₋ₓO_{y}), transitional metal oxides such as ABO where A is a transitional metal such as Fe, Co, Ni, B is a transitional, inert or light metal such as AI, Ta, Pt, Hf, or Cr in all constituting and alloys such as CoTaPtCr, FeCoTaPt, AlNiCoTa, carbides such as RCoC where R can be Y, Gd, Er, Lu, and/or Ta, and mixtures, laminates, or combinations thereof.

The components and structures of the invention can be constructed or fabricated by, for example, deposition processes, such as, but not limited to, physical vapor deposition, DC-magnetron sputtering, radio frequency sputtering, pulsed laser deposition, evaporation, physical vapor transport, and molecular beam epitaxy, chemical vapor deposition processes including metallorganic chemical vapor deposition (MOCVD), organometallic vapor phase epitaxy (OMVPE), chemical vapor deposition (CVD), chemical vapor transport (CVT), plasma assisted/enhanced, and liquid and gel state deposition process including liquid phase epitaxy (LPE), solvus-thermal processes such as hydrothermal and ammonothermal deposition and sol-gel processes; electrochemical deposition processes including electrolysis, electro-deposition and electroplating.

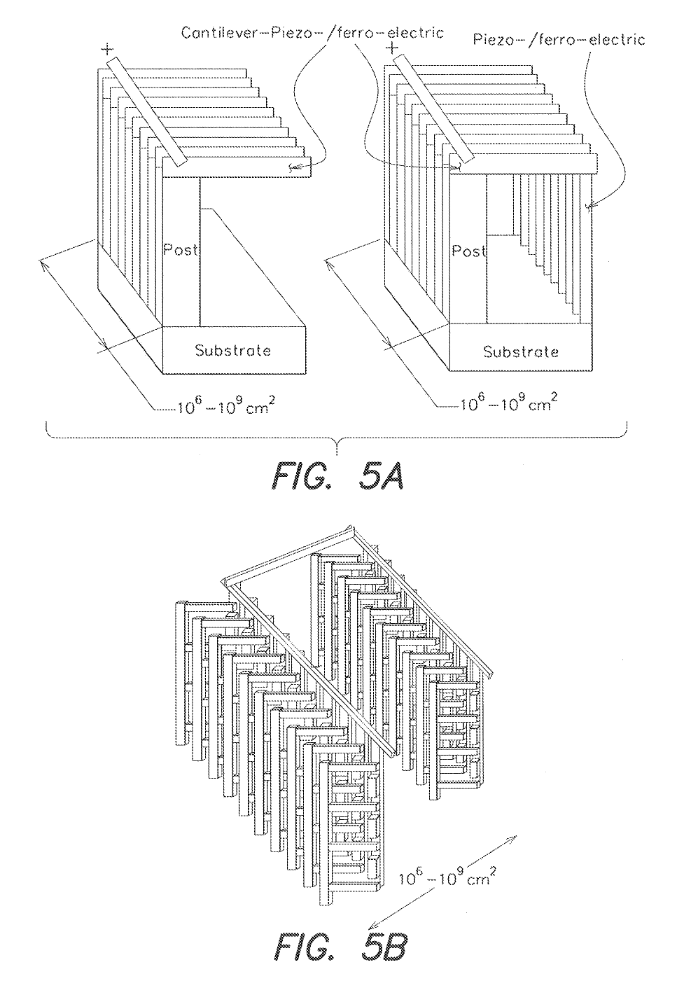

A plurality of components or structures can be utilized and be collectively or selectively electrically connected. For example, one or more charge-generating assemblies may be serially connected or be connected in parallel as schematically illustrated in FIGS. 5A and 5B. The cumulative energy can then be aggregated and stored in the one or more storage structures.

Any one or more of the devices can also be coated with a scar inhibitor such as steroids including, but not limited to dexamethasone sodium phosphate.

Other aspects of the invention are directed to a method of electrophysiologic stimulation. In some embodiments pertinent to such aspects of the invention, the method of electrophysiologic stimulation can comprise identifying a patient susceptible to arrhythmic cardiac pacing, and implanting at least one pacing device 100 in the patient. The at least one implanted pacing device can comprise at least one charging circuit 200 configured to store at least a portion of electrical energy generated from at least one of a piezoelectric and a magnetostrictive material or similar compounds that provide or utilize Lorentzian effects. Device 100 can also comprise at least one pulse-generating circuit 120 configured to deliver at least a portion of the stored electrical energy in at least one electrophysiologic stimulating charge 154 comprising at least a portion of stored energy 151 to at least one cardiac chamber to heart H of the patient. Implanting device 200 can comprise securing dynamic or oscillating member 220 to a first tissue region D of a pulsating organ and securing the stationary member 220 or an end of component 200 to a second tissue region S. The method can further comprise implanting a plurality of the devices in the patient. For example, the generated electric potential over a plurality of cyclic events is typically stored at a level sufficient to correct, for example, Brady-arrhythmic disorders or pace the heart. The delivered electrical energy or pacing charge 154 can stimulate excitable cells in the cardiac tissue by producing a self-propagating wave front of action potentials sufficient to result in tissue contraction.

The devices and techniques of the invention can also pertain to the fabrication and utility of nanoscale and microscale component that can interface, directly or indirectly, with myocardial tissue or any tissue within or without the cardiac surface by way of one or more microscale or nanoscale electrodes 600. Preferably, the one or more electrodes 600 are disposed to be within about 20 nm to about 5 μm to at least one pacing device. In some cases, the electrode is disposed on or adjacent the surface or body 110 of the charge generating device. For example, the electrodes which ultimately delivers the pacing power (I*V) may be fabricated through microelectronic processes to form nanostructures or microstructures deposited on the back surface of the device or the surface of the device in direct contact with the myocardium or any tissue cable of pacing in such a manner that lengthy and macroscale electrical leads are eliminated.

Electrode 600 can comprise a biologically inert material such as titanium, silver, metallic alloys, a semi-metal, or a semiconductor.

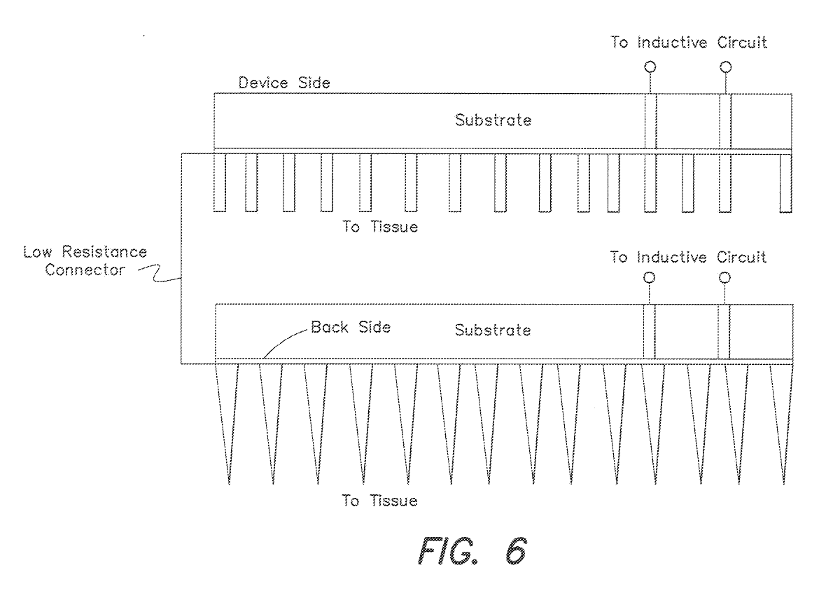

FIG. 6 depicts an exemplary electrode of the invention that is capable of delivering an electric potential for one or more pacing events. The electrodes can comprise biologically inert metallic posts deposited by chemical or physical vapor deposition or by electrochemical methods on to the backside of the substrate material bearing the frequency responsive materials and assemblies of the invention ranging in size from about 20 nm to about 50 mm in length and from about 20 nm to about 200 μm in diameter. In accordance with preferred embodiments of the invention, the electrodes are placed in direct contact with the posterior of the heart surface.

Affixation of the novel device or components thereof to the myocardium or other tissue can be performed by use of metallic screws, or by surgical suture with, for example, antibacterial threads. In other cases, anchoring structures comprising one or more barbed features may sufficiently secure the one or more devices or portions thereof to tissue. For example, the oscillating end of generator 200 may be secured to tissue at position D by one or more extending barbs and the static end of generator 200 may be secured to tissue at position S by one or more screws.

The one or more reflexive sensing circuit 130 can detect arrhythmia and normal electrical impulse. In some embodiments of the invention, the failure circuit can comprise an effective solid state capacitance ranging from about 10 pF/cm² to about 100 mF/cm² defined as the nominal capacitance; effective resistor value not exceeding about 50 KOhms and a rectifying diode. The failure circuit can be connected to a gate terminal of a metal oxide semiconductor field effect transistor (MOSFET), which can be a p-type MOSFET or PMOSFET and supplies a gate voltage, V_{g}. The pacing circuit can comprise an effective solid state capacitance ranging from about 10 pF/cm² to about 100 mF/cm² and a rectifying diode such that the pacing circuit is connected to the source terminal of the MOSFET and supplies a source voltage, Vₛ. In further embodiments, the can utilize an inductor circuit comprising, for example, primary and secondary inductance coils wherein the inductance of the secondary coil exceeds the inductance of the primary coil. The drain terminal of the MOSFET is preferably connected to the inductive circuit.

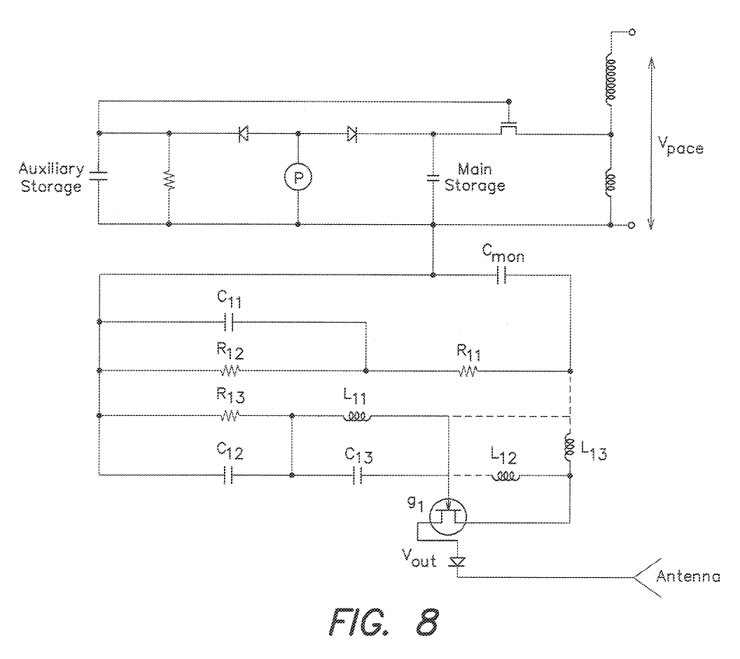

During normal mode of operation of the heart, the frequency response of the materials of the invention typically creates a charge which is stored in the effective capacitor bank comprising one or more capacitors of effective/cumulative capacitance C_{eff}, within the range of the nominal capacitance of both the failure and pacing circuit. In preferred configurations, the failure circuit is connected to a “normally-off” MOSFET and no current flow or potential drop is allowed from the pacing circuit. The circuits of the devices can also be configured such that the charge generated by individual or discrete pulsation events is insufficient to supply the requisite gate voltage, V_{g}, to turn the transistor on. However, the charge generated by each discrete pulsation is stored in, for example, a failure capacitor bank, denoted as “Auxiliary Storage” in the exemplary failure circuit of FIG. 8. Effectively, the MOSFET can act as a switch which regulates the activities of the failure and pacing circuits.

When the natural pulsation of the heart seizes, no charge follows from the materials of invention or the power source through the failure diode into Auxiliary Storage. This can create a potential drop through the resistor in the failure circuit, which corresponds to detecting a failure condition. A potential would then be created and applied at the gate of the transistor of, for example, at least the magnitude of V_{g}, which activates the transistor for the duration of discharge of Auxiliary Storage. Because of the application of V_{g}, current can flow from the capacitor in the pacing circuit labeled “Main Storage” through the channel of the MOSFET to the drain terminal of the MOSFET, which is preferably connected to the inductive circuit for the duration of the discharge of the capacitor labeled “Main Storage”. The inductive circuit typically utilizes primary and secondary inductances to build a voltage of about 1 V/s which can be delivered to cardiac tissue, thereby effecting reflex stimulation in accordance with some aspects of the invention. In effect, the cycle completes the detection of heart failure and the pacing enabled by one circuit.

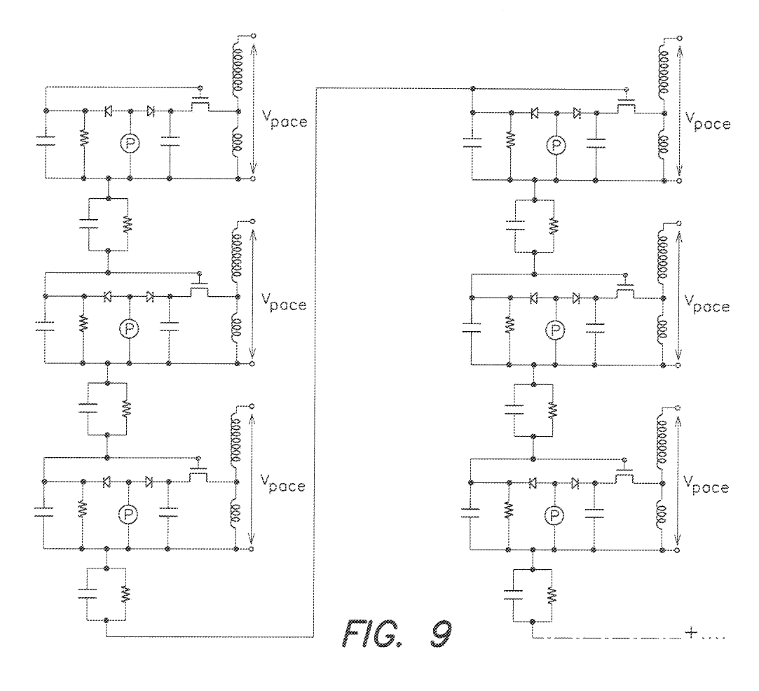

As exemplarily shown in FIG. 9, some embodiments of the invention can comprise configurations involving a plurality of discrete circuits, such as the embodiments illustrated in FIG. 8, preferably connected by a time delay circuit that can facilitate multiple stimulation events over a period of time of value, N. τ, where N is the number of circuits and τ is about 1/R_{d}C_{d} which is typically the time delay facilitated by the delay resistances and capacitance, R_{d} and C_{d}, respectively. The delay is preferably within the range 10 nanoseconds≦τ≦5 seconds. The delay capacitance can be similarly charged by the materials of invention and connected to the failure circuit. In the multiple circuit configurations, after the pacing of the first circuit and first delay expires, pacing or stimulation by subsequent circuits or devices can occur.

Multiple pacing devices advantageously disposed at different locations in any one or more of the myocardium, ventricular tissues, and atrial tissues can facilitate pacing with a plurality of pacing charges that can provide a plurality of stimuli at a plurality of locations, which can improve overall pacing efficacy.

Non-invasive or minimally invasive delivery of the devices of the invention at particular positions and orientations within a patient can be performed by the use of a catheter with sufficient internal dimensions to accommodate devices having, preferably, an outer diameter of less than about 1 inch, more preferably, less than about 2 cm.

Communication between two or more of the implanted or devices and/or to systems external systems can be established through opto-electronic circuit components such as a photodetector and a laser, integrated onto the each device, or by use of radio-frequency circuit components, such as a wireless rf-transmitter and a receiver comprising, but not limited to, device components such field effect transistors, bi-polar junction transistors, diodes, capacitors and inductors. For example, the devices of the invention as illustrated in FIG. 8 can comprise subsystems having a failure circuit, a pacing circuit, and an inductive circuit, as well as one or more communication subsystems comprising one or more transmission circuits and one or more receiving circuits. The one or more communication subsystems can be is designed to transmit at between 1 KHz and 500 MHz through a transmitter utilizing, for example, any of a Colpitts, Hartley, Clapp, Armstrong, and Vackar transmitter or a combination thereof. The Colpitts transmitter circuit as exemplarily illustrated in FIG. 8 can transmit, to, for example, the posterior of the patient's body, the potential drop across a monitoring capacitor, Cₘₒₙ, of capacitance within range of the nominal capacitance which typically represents the magnitude of the capacitance across the Main Storage and Auxiliary Storage capacitances through voltage dividers with capacitances C₁₁, and C₁₂, and resistors R₁₁, R₁₂ and R₁₃, which typically do not exceed 100 KOhms. An LC circuit with capacitance C₁₃ and inductance L₁₁ with an inductance tank having one or more inductors as (shown as the dotted circuit extension) can comprise inductances L₁₂ and L₁₃ and an active gain component, such as a bipolar junction transistor and/or a field effect transistor and/or a diode, can be utilized to boost the range and gain, g₁, of the transmitter to communicate an output voltage, Vₒᵤₜ through the output diode and Antenna, shown in FIG. 8. A receiver with an operating frequency in the range of from about 1 KHz to about 500 MHz can be utilized to receive the one or more transmitted signals, thus providing or determining the potential of Cₘₒₙ.

An aspect of the invention may pertain to the integration of flows sensors to devices consisting of flow sensors such as a microfluidic sensor capable of determining flow and turbulence with ventricular or atrial chambers. Pressure sensors may be utilized with one or more devices of the invention with one or more pressure sensors such as a microfluidic sensor capable of determining the pressure of fluid within ventricular or atrial chambers.

Device 100 may utilize one or more processor or may include specially-programmed, special-purpose hardware, for example, an application-specific integrated circuit (ASIC). Device 100 can include one or more processors typically connected to one or more memory devices or structures, which can comprise, for example, any one or more of flash memory devices, RAM memory devices, or other data storage apparatus. The one or more memory devices can be used for storing programs and data during operation of the device 100. For example, the memory may be used for storing historical data relating to the operating parameters over a period of time, as well as operating data.

The function and advantages of these and other embodiments of the invention can be further understood from the example below, which illustrates the benefits and/or advantages of the one or more systems and techniques of the invention but do not exemplify the full scope of the invention.

### EXAMPLE

This example prophetically describes a cardiac pacing device in accordance with some embodiments of the invention.

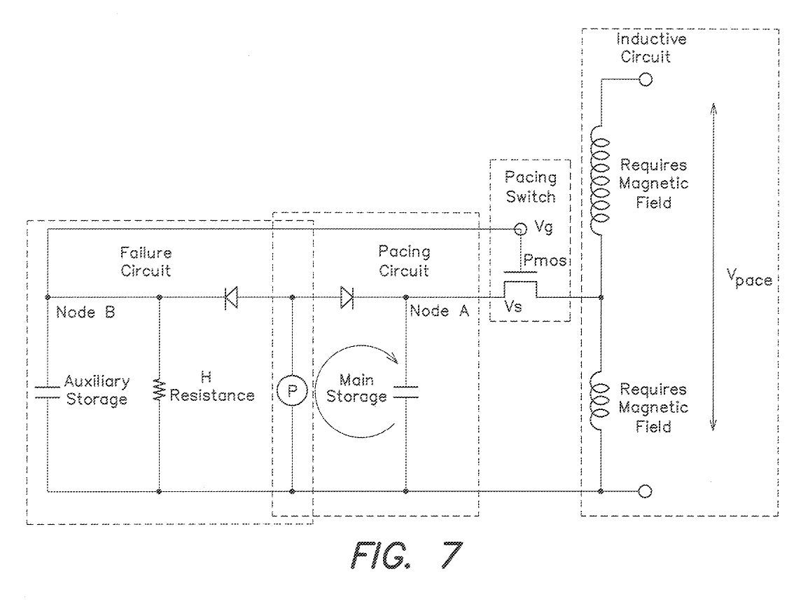

The device can have a sensing or failure sub-circuit comprising a rectifying component with one or more diodes connected in series with an auxiliary charge storage component with one or more capacitive components and one or more resistive components connected to one or more power sources, as shown exemplarily illustrated in FIG. 7.

The resistive component can have a high resistance ranging from about 10 mOhms to about 100 KOhms.

The device can also have one or more pacing subcircuits comprising at least one rectifying component of diodes connected in series with one or more of the charge storage components, and one or more resistive components connected to one or more power sources.

The device can also comprise one or more transformer circuits comprising at least one inductive component with one or more primary inductors and/or one or more secondary inductors and, optionally, magnetic or ferroelectric materials.

The device can also comprise a switching component or circuit to which the various sub-circuits could be connected, and comprising a switching electronic device such as a transistor such as, but not limited to, field effect or bipolar junction transistors, and optionally may activate the inductive circuit. The switching device can be a metal on insulator/oxide semiconductor field effect transistor.

Having now described some illustrative embodiments of the invention, it should be apparent to those skilled in the art that the foregoing is merely illustrative and not limiting, having been presented by way of example only. Numerous modifications and other embodiments are within the scope of one of ordinary skill in the art and are contemplated as falling within the scope of the invention. In particular, although many of the examples presented herein involve specific combinations of method acts or system elements, it should be understood that those acts and those elements may be combined in other ways to accomplish the same objectives.

Those skilled in the art should appreciate that the parameters and configurations described herein are exemplary and that actual parameters and/or configurations will depend on the specific application in which the systems and techniques of the invention are used. Those skilled in the art should also recognize or be able to ascertain, using no more than routine experimentation, equivalents to the specific embodiments of the invention. It is therefore to be understood that the embodiments described herein are presented by way of example only and that, within the scope of the appended claims and equivalents thereto; the invention may be practiced otherwise than as specifically described.

Moreover, it should also be appreciated that the invention is directed to each feature, system, subsystem, or technique described herein and any combination of two or more features, systems, subsystems, or techniques described herein and any combination of two or more features, systems, subsystems, and/or methods, if such features, systems, subsystems, and techniques are not mutually inconsistent, is considered to be within the scope of the invention as embodied in the claims. Further, acts, elements, and features discussed only in connection with one embodiment are not intended to be excluded from a similar role in other embodiments. Indeed, other aspects of the invention can be directed to modifying or supplementing existing implanted devices such as ICDs. For example, any one or more of the various devices and techniques disclosed herein can be implanted and connected to existing ICDs to provide a trigger that stimulates or activates the one or more devices of the invention. In some further aspects, the ICD which are currently commercially available and easy or at least have known implanting techniques, could supply the “heart beat” or triggering condition for the reflex pacers, which can be implanted at more difficult sites relative to conventionally reached sites in the heart. But because the devices and techniques of the invention are typically not dependent on the primary pacemaker for energy—they can perpetually pace which can produce facilitate multi-site pacing. Further, existing devices could be made less complex devices, and consequently cheaper, by utilizing autonomous reflex pacing devices. There would also be little or no concern for safety issues and the devices can retain a very small size without a large storage capacitor, i.e., battery.

As used herein, the term “plurality” refers to two or more items or components. The terms “comprising,” “including,” “carrying,” “having,” “containing,” and “involving,” whether in the written description or the claims and the like, are open-ended terms, i.e., to mean “including but not limited to.” Thus, the use of such terms is meant to encompass the items listed thereafter, and equivalents thereof, as well as additional items. Only the transitional phrases “consisting of” and “consisting essentially of,” are closed or semi-closed transitional phrases, respectively, with respect to the claims. Use of ordinal terms such as “first,” “second,” “third,” and the like in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

What is claimed is:

1. An implantable cardiac device, comprising:
a charging circuit configured to store electrical energy generated from at least one of a piezoelectric material and a magnetostrictive material; and
a pulse-generating circuit operatively coupled to the charging circuit and configured to deliver at least a portion of the stored electrical energy in at least one electrical pacing charge to at least one cardiac chamber.

2. The implantable cardiac device of claim 1, wherein the charging circuit comprises at least one storage device.

3. The implantable cardiac device of claim 2, wherein the at least one storage device comprises at least one capacitor.

4. The implantable cardiac device of claim 1, further comprising a sensing circuit configured to monitor cardiac activity of at least one cardiac chamber and energize the pulse-generating circuit to deliver the at least one electrical pacing charge to the at least one cardiac chamber when the monitored cardiac activity comprises at least one arrhythmic cardiac condition.

5. The implantable cardiac device of claim 4, wherein the at least one arrhythmic cardiac condition is selected from the group consisting of tachy-arrhythmia, brady-arrhythmia, abnormal inter-atrial conduction, and asynchronous ventricular activation.

6. The implantable cardiac device of claim 1, wherein the charging circuit comprises an electrical generator having a cantilever element comprising the at least one of the piezoelectric and magnetostrictive material operatively engaged with a rigid element.

7. The implantable cardiac device of claim 6, wherein the at least one of the piezoelectric and magnetostrictive material comprises a compound selected from the group consisting of zinc oxide, gallium nitride, cuprates, titanates, related alloys, and mixtures thereof.

8. A method of electrophysiologic stimulation, comprising:
identifying a patient susceptible to arrhythmic cardiac pacing; and
implanting a cardiac pacing device in the patient, the cardiac pacing device comprising at least one charging circuit configured to store electrical energy generated from at least one of a piezoelectric and a magnetostrictive material, and a pulse-generating circuit configured to deliver at least a portion of the stored electrical energy in at least one electrophysiologic stimulating charge to at least one cardiac chamber.

9. The method of claim 8, wherein the cardiac pacing device comprises a sensing circuit configured to monitor cardiac activity of at least one cardiac chamber and energize the pulse-generating circuit to deliver the at least one electrophysiologic stimulating charge.

10. The method of claim 9, wherein the sensing circuit is configured to energize the pulse-generating circuit when at least one arrhythmic cardiac condition selected from the group consisting of tachy-arrhythmia, brady-arrhythmia, abnormal inter-atrial conduction, and asynchronous ventricular activation is identified.

11. The method of claim 9, wherein the charging circuit comprises at least one generator, each of the at least one generator having at least one oscillating member, comprising the at least one of the piezoelectric and the magnetostrictive material, at least one stationary member in corresponding engagement with the at least one oscillating member, and a dielectric material disposed between the correspondingly engaged at least one oscillating member and the at least one stationary member.

12. The method of claim 11, wherein implanting the cardiac pacing device comprises securing the oscillating member to a first tissue region of a pulsating organ and securing the stationary member to a second tissue region of the pulsating organ.

13. The method of claim 11, further comprising implanting a plurality of the cardiac pacing devices in the patient.
